(19) United States
(12) Patent Application Publication
(10) Pub. No.: US 2003/0124712 A1
(43) Pub. Date: Jul. 3, 2003

(54) Method and apparatus for differentiating articles in a product stream

(76) Inventors: Mark A. Bauman, College Place, WA (US); Timothy L. Justice, Walla Walla, WA (US); Maurice J. Hunking JR., Walla Walla, WA (US)

Correspondence Address: WELLS ST. JOHN ROBERTS GREGORY & MATKIN P.S., 601 W. FIRST AVENUE, SUITE 1300, SPOKANE, WA, 99201-3828, US

(21) Appl. No.: 10/038,425

(22) Filed: Jan. 2, 2002

(51) Int. Cl. C12M 1/00

(52) U.S. Cl. 435/283.1

(57) ABSTRACT

An apparatus and method for differentiating articles in a product stream is described, and which includes an acoustic generator for generating acoustic energy; a plurality of articles located in receiving relation relative to the acoustic energy, and wherein the plurality of articles receive at least a portion of the acoustic energy, and wherein at least some of the acoustic energy received by the plurality of articles is converted into electromagnetic energy; a sensor for detecting at least some of the electromagnetic energy generated by the conversion of the acoustic energy; and a processor operably coupled with the sensor for differentiating the plurality of articles based upon the electromagnetic energy sensed.

### TECHNICAL FIELD

The present invention relates to a method and apparatus for differentiating articles in a product stream.

### BACKGROUND OF THE INVENTION

In industry, various inspection devices are used to differentiate desirable and undesirable articles in a product stream. Such inspection devices are often utilized to differentiate materials based on the presence of impurities, to evaluate quality and to help ensure safety. These inspection devices have often been incorporated into sorting systems, which are then able to facilitate both the inspection of articles in a product stream, and the subsequent sorting of the acceptable and unacceptable articles. Inspection and sorting systems such as these may be used in a variety of applications. For example, in the food products industry, such devices are used to differentiate between food products which are acceptable for human consumption and those which are unacceptable for human consumption.

Although a variety of inspection devices have been used with varying degrees of success, none of these inspection devices have been able to adequately differentiate between acceptable and unacceptable articles in difficult applications, where there are no obvious measurable attributes by which to differentiate the acceptable and unacceptable articles. One example of such a difficult application is the sorting of chicken meat, from chicken bone and cartilage fragments. In this difficult application, X-ray systems have been used with limited success heretofore. Such X-ray systems rely on differences in material density to differentiate between the desired chicken meat, and the undesired bones and cartilage. Unfortunately, because the material densities of chicken meat, bones and cartilage may be quite similar, some fragments of bone and cartilage go undetected. This is especially true when young chickens are processed. Such undetected fragments of bone and cartilage in “boneless” chicken are not only undesirable to the manufacturer, they are also dangerous to the consumer. For example, when a consumer eats a portion of “boneless” chicken which contains a hidden fragment of bone and cartilage, the fragment may cause the consumer to break a tooth, or may even lodge in the consumer's throat, causing the consumer to choke on the offending fragment.

Other techniques have also been considered as the search for a method and apparatus which can adequately differentiate desired chicken meat, and the undesired bone and cartilage continues. For example, methods such as nuclear magnetic resonance have been considered. However, nuclear magnetic resonance systems have not been adopted due to their slow speed and prohibitive costs. Such systems are also unacceptable because current commercially available systems are unable to operate at production line speeds. Furthermore, techniques such as traditional ultrasound and other acoustic systems have been tried, but the ability of these systems to adequately differentiate the desired and undesired articles has been less than satisfactory.

These and other shortcomings in the prior art devices and practices are addressed by means of the present invention which is discussed in further detail below.

### SUMMARY OF THE INVENTION

One aspect of the present invention is to provide an apparatus for differentiating articles, and which includes an acoustic generator for generating acoustic energy; a plurality of articles located in receiving relation relative to the acoustic energy, and wherein the plurality of articles receive at least a portion of the acoustic energy, and wherein at least some of the acoustic energy received by the plurality of articles is converted into electromagnetic energy; a sensor for detecting at least some of the electromagnetic energy generated by the conversion of the acoustic energy; and a processor operably coupled with the sensor for differentiating the plurality of articles based upon the electromagnetic energy sensed.

Another aspect of the present invention is to provide an apparatus for differentiating articles in a product stream, and which includes an acoustic generator for generating acoustic energy; a product stream located in receiving relation relative to the acoustic energy, and wherein the product stream includes a plurality of articles, and wherein at least some of the acoustic energy is received by the plurality of articles, and wherein at least some of the acoustic energy received by the plurality of articles is converted into electromagnetic energy; a sensor for detecting at least some of the electromagnetic energy generated by the conversion; and a processor operably coupled with the sensor for differentiating the plurality of articles.

A further aspect of the present invention is to provide an apparatus for differentiating articles in a product stream, and which includes a product stream which includes acceptable and unacceptable articles with differing electromechanical conversion efficiencies; an acoustic generator which produces acoustic energy which is directed to the articles traveling in the product stream, and wherein the respective articles based upon their individual electromechanical conversion efficiencies convert the acoustic energy into electromagnetic energy; a sensor for receiving at least some of the electromagnetic energy generated by the conversion of the acoustic energy; and a processor operably coupled with the sensor for differentiating between the acceptable and unacceptable articles based on their respective electromechanical conversion efficiencies.

A further aspect of the present invention is to provide an apparatus for differentiating acceptable and unacceptable articles in a product stream, and which includes an acoustic generator for generating acoustic energy; a product stream located in receiving relation relative to the acoustic energy, and which may include acceptable articles including meat and unacceptable articles including bone and cartilage, and wherein the acceptable and unacceptable articles have differing electromechanical conversion efficiencies, and wherein the acceptable and unacceptable articles receive at least some of the acoustic energy, and wherein the acceptable and unacceptable articles convert at least some of the received acoustic energy into electromagnetic energy; a sensor for detecting at least some of the electromagnetic energy generated by the conversion; and a processor operably coupled with the sensor for differentiating between the acceptable and unacceptable articles based on their respective electromechanical conversion efficiencies.

Yet a further aspect of the present invention is to provide an apparatus for differentiating acceptable and unacceptable articles in a product stream, and which includes an acoustic generator including a plurality of transducers positioned at predetermined angles relative to the product stream for generating acoustic energy; a product stream located in receiving relation relative to the acoustic energy, and wherein the product stream includes acceptable articles consisting of meat and unacceptable articles which include bone and cartilage, and wherein the acceptable and unacceptable articles have differing electromechanical conversion efficiencies, and wherein the acceptable and unacceptable articles receive at least some of the acoustic energy, and wherein the acceptable and unacceptable articles convert at least some of the received acoustic energy into electromagnetic energy; a sensor for detecting at least some of the electromagnetic energy generated by the conversion of the acoustic energy; and a processor operably coupled with the sensor, and which utilizes information received from the sensor to map the electromechanical conversion efficiencies of the acceptable and unacceptable articles, and to differentiate between the acceptable and unacceptable articles.

Still a further aspect of the present invention is to provide a method for differentiating articles, and which includes generating acoustic energy; directing the acoustic energy toward a product stream which includes acceptable and unacceptable articles having differing electromechanical conversion efficiencies; converting at least some of the acoustic energy received by the acceptable and unacceptable articles into electromagnetic energy at rates relative to the acceptable and unacceptable articles respective electromechanical conversion efficiencies; detecting at least some of the electromagnetic energy generated by the conversion; and differentiating the acceptable and unacceptable articles based upon their respective electromechanical conversion efficiencies as indicated by the sensed electromagnetic energy.

### BRIEF DESCRIPTION OF THE DRAWINGS

Preferred embodiments of the invention are described below with reference to the following accompanying drawings.

FIG. 1 is a diagrammatic, fragmentary view of a sorting system incorporating the apparatus of the present invention.

FIG. 2 is a block diagram representing an inspection station employed with the present invention.

FIG. 3 is a block diagram representing a main computer and micro-controller board employed with the present invention.

FIG. 4 is a diagrammatic, transverse sectional view of a detection chamber employed with the present invention.

FIG. 5 is a diagrammatic, fragmentary side elevational view of the detection chamber of the present invention.

FIG. 6 is a diagrammatic, fragmentary, sectional view of an electromagnetic sensor utilized in the present invention.

FIG. 7 is a block diagram representing a electromagnetic sensor employed with the present invention.

FIG. 8 is a schematic diagram showing an electromagnetic sensor arrangement employed with the present invention.

FIG. 9 is a block diagram showing an acoustic generator employed with the present invention.

FIG. 10 is a block diagram showing a signal processor employed with the present invention.

FIG. 11 is a graphical depiction illustrating an existing timing relationship utilized with the present invention.

FIG. 12 is a flow-chart representing the operation of the present invention.

FIG. 13 is a diagrammatic representation illustrating the interaction of acoustic and electromagnetic energy on an object of interest.

FIG. 14 is a drawing illustrating the timing and sequencing of the acoustic energy, and the relative response of the converted electromagnetic energy generated by an article of interest.

### DETAILED DESCRIPTION OF THE PREFERRED EMBODIMENTS

This disclosure of the invention is submitted in furtherance of the constitutional purposes of the U.S. Patent Laws “to promote the progress of science and useful arts” (Article 1, Section 8).

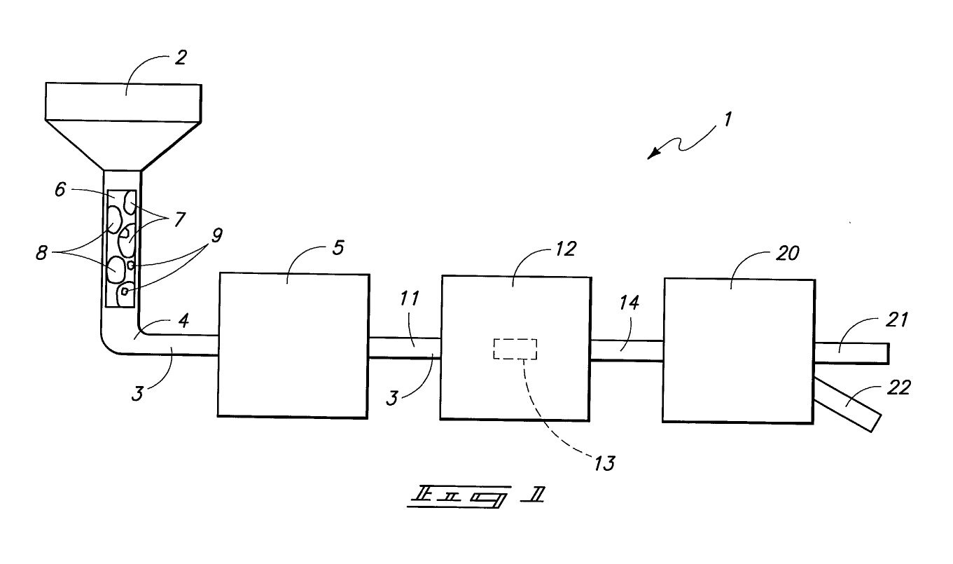

Referring now to FIG. 1, a diagrammatic view of a device which incorporates the apparatus of the present invention is generally indicated by the numeral 1. The device 1 includes a product hopper 2 for receiving product or articles to be inspected and queuing the product or articles into a resulting stream such that they can be inspected and subsequently sorted as to given characteristics.

Again referring to FIG. 1, a transport pipe 3 is shown, and which extends from the product hopper 2. The transport pipe 3 includes a first section 4 which couples the product hopper 2, and a pump 5 in fluid flowing relation. The pump 5 is of a suitable design to minimize damage to the product or articles traveling in the stream, and further functions to move the product stream through the transport pipe 3 under a predetermined pressure. Such pumps are well known in the industry and are widely available for pumping a wide range of products.

As seen in FIG. 1, a portion of the first section 4 of transport pipe 3 has been removed to reveal the product stream 6 moving therein. The product stream 6 includes a plurality of articles 7 which are to be differentiated and then sorted as to a predetermined characteristic. The product stream 6 may include both acceptable and unacceptable articles 8 and 9. By way of example only, the product stream 6 may include food products comprising acceptable articles 8 including boneless chicken meat, and unacceptable articles 9 including bone or cartilage fragments. However, the device 1 may also be used in other applications. For example, the device 1 could be used to sort a slurry of fluids including acceptable and unacceptable minerals, or any other materials which are to be differentiated as to type by means of the teachings of this document.

A second section 11 of transport pipe 3 extends between the pump 5 and an inspection station 12. This second section of transport pipe 11 couples the pump 5 and the inspection station 12 in fluid flowing relation. Referring still to FIG. 1, a section of transport pipe 3 which constitutes a differentiation section is indicated by the numeral 13 (shown in phantom lines). The differentiation section 13 is located within the inspection station 12. As the product stream 6 flows through the differentiation section 13, the inspection station 12 functions to differentiate or otherwise identify the acceptable and unacceptable articles 8 and 9 in the product stream 6.

Again referring to FIG. 1, a third section 14 of the transport pipe 3 exits the inspection station 12. This third section 14 of the transport pipe 3 couples the inspection station 12 in fluid flowing relation relative to a product ejector 20. The product ejector 20 directs the acceptable articles 8 into an acceptance pipe 21, and directs the rejected or unacceptable article 9 into a rejection pipe 22.

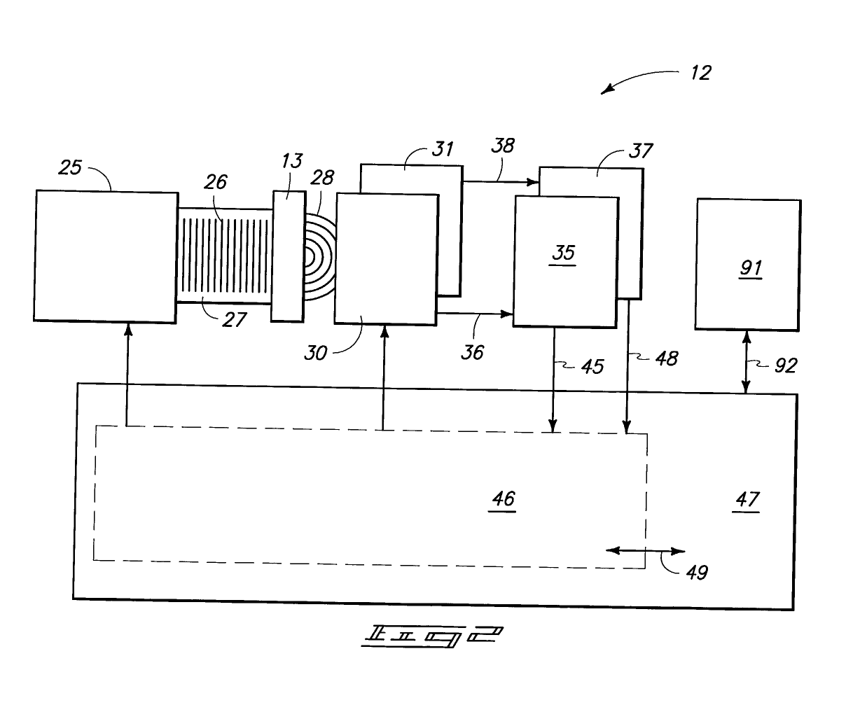

Referring now to FIG. 2, the inspection station 12 of the present invention is shown in a block diagram. As shown in FIG. 2, an acoustic generator 25 generates acoustic energy 26. This acoustic energy 26, once emitted, travels through an acoustic medium 27, and is directed by same towards the product stream (not shown) which is flowing through the differentiation section 13. A portion of the acoustic energy 26 is then received by each of several articles in the product steam. Each of the several articles which received a portion of the acoustic energy 26 then converts at least some of the received acoustic energy into electromagnetic energy 28. Each of the articles in the product stream converts the acoustic energy 26 into electromagnetic energy 28 at a rate which is relative to each articles respective electromechanical conversion properties.

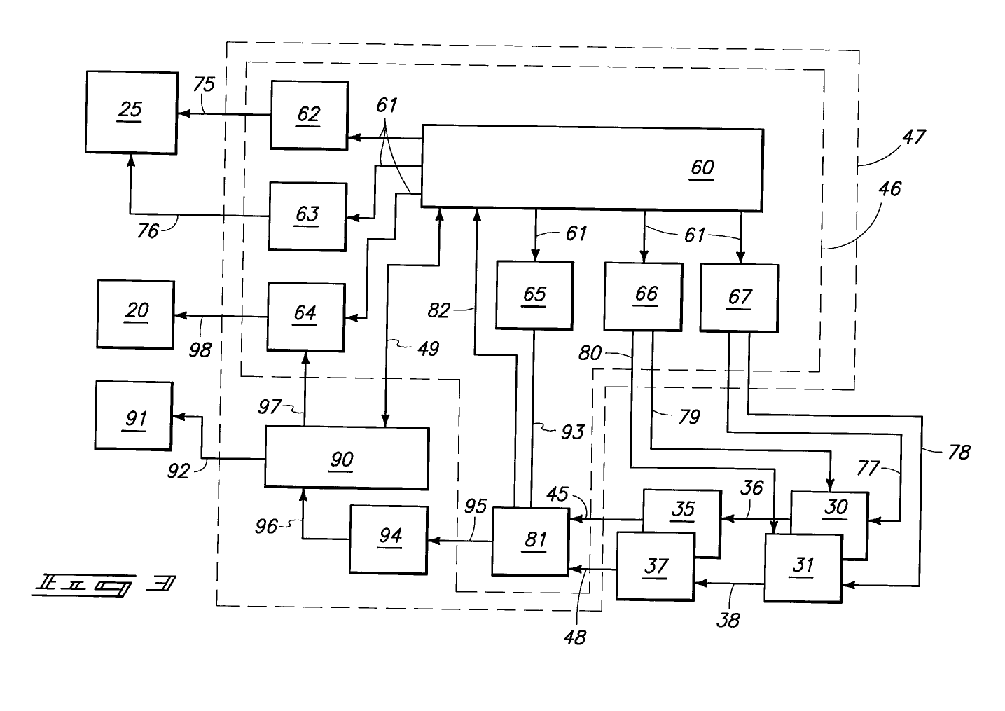

Referring again to FIG. 2, the electromagnetic energy 28 produced by the conversion of the acoustic energy 26 is detected by the first and second electromagnetic sensors 30 and 31. These two electromagnetic sensors 30 and 31 function to receive, amplify, and convert the electromagnetic energy 28 which was generated by the articles in the product stream. The first electromagnetic sensor 30 is electrically coupled to a first signal processor board 35 by a first signal path 36. The second electromagnetic sensor 31 is similarly electrically coupled to the second signal processor board 37 by a second signal path 38. A first processed signal path 45 is provided, and which carries demodulated output signals from the first signal processor board 35 to a processor comprising a micro-controller board 46. The second processed signal path 48 similarly carries demodulated output signals from the second signal processor board 37 to the micro-controller board 46. A data link 49 is provided, and which electrically couples the micro-controller board 46 to the main computer 47. More specifically, data link 49 electrically couples the micro-controller board 46 to the main computer's CPU 90 (FIG. 3). In a preferred embodiment, the micro-controller board 46 is positioned within the main computer 47 and data is transferred by utilizing shared memory and a PCI bus which is not shown. A conventional power supply (not shown) provides the inspection station with appropriate amounts of electrical power.

Now referring to FIG. 3, a block diagram is provided which illustrates the various electrical couplings of the main computer 47 and micro-controller board 46. As shown in FIG. 3, the micro-controller board 46, and the main computer 47, act together to coordinate the operation of the inspection station 12. The micro-controller board 46, as a general matter, typically controls real-time tasks, while the main computer 47 performs the more computationally intensive, but less time sensitive tasks. During initialization, a program is downloaded from the main computer 47, and is executed by the micro-controller board CPU indicated by the numeral 60. A commercially available micro-controller board may be secured from National Instruments as model 7030 data acquisition board with an on-board micro-controller CPU which is a 133 MHz AMD 486DX5.

Referring again to FIG. 3, the micro-controller CPU 60 is shown to have a plurality of electrical outputs 61 which electrically couple the micro-controller CPU 60 respectively to the burst timing circuit 62; the transducer selection circuit 63; the ejector timing circuit 64; the analog to digital timing circuit 65; the gate calibration circuit 66; and the mixer timing circuit 67. Each of these circuits are located on the micro-controller board 46.

As best seen in FIG. 3, the burst timing circuit 62 is electrically coupled to the acoustic generator 25 by the burst enable electrical link 75. Further, the transducer selection circuit 63 is electrically coupled to the acoustic generator 25 by the transducer selector link 76.

Referring still to FIG. 3, the mixer timing circuit 67 is electrically coupled to the first electromagnetic sensor 30 by the first mixer enable link 77, and is similarly electrically coupled to the second electromagnetic sensor 31 by the second mixer enable link 78. The first and second electromagnetic sensors 30 and 31 utilize the mixer timing circuit 67 to up-convert the signal to prevent overloading the system during periods when the acoustic generator 25 is emitting acoustic energy. An accurate tuning of the first and second electromagnetic sensors 30 and 31 is required for proper operation of device 1. In this regard, the gate calibration circuit 66 is responsible for accurately tuning the first and second electromagnetic sensors 30 and 31. For example, the gate calibration circuit 66 develops control voltages which are sent to the first and second electromagnetic sensors 30 and 31 to tune the input tank of same to resonance at the burst frequency. The gate calibration circuit 66 is also electrically coupled to the first electromagnetic sensor 30 by the first electromagnetic senor link 79, and is similarly electrically coupled to the second electromagnetic sensor 31 by the second electromagnetic sensor link 80. The gate calibration circuit 66 is also operably coupled with the micro-controller CPU 60. The micro-controller board CPU 60 monitors electrical data received from the on-board analog-to-digital converter 81 during calibration, and makes adjustments accordingly. As seen in FIG. 3, the analog-to-digital converter 81 is electrically coupled to the micro-controller CPU 60 by way of the micro-controller data link 82. The gate calibration circuit 66 is contained on the micro-controller board 46, and has a 16 bit resolution, with an output range of not greater than about 10 volts.

Still referring to FIG. 3, the main computer CPU 90, is electrically coupled to an operator interface 91 by an operator interface link 92. In a preferred embodiment, an Intel Pentium III class industrial PCI computer which includes a means of coupling to the micro-controller board, and electrical connections for an external touchscreen are utilized. An external touchscreen serves as the operator interface 91.

Referring to FIG. 3, the analog-to-digital conversion timing circuit 65 is electrically coupled to the analog-to-digital converter 81 by a converter link 93. The analog-to-digital converter 81 is also electrically coupled to the micro-controller CPU 60. Both the analog-to-digital conversion timing circuit 65, and the analog-to-digital converter 81 are positioned on the micro-controller board 46.

Referring still to FIG. 3, demodulated signals from the first signal processor board 35 are delivered to the analog-to-digital converter 81 by the first processed signal path 45. Similarly, demodulated signals from the second signal processor board 37 are delivered to the analog-to-digital converter 81 by the second processed signal path 48. Analog-to-digital converter 81 is located on the micro-controller board 46. The analog-to-digital converter 81 is electrically coupled to a waveform analysis and correlation unit 94 by a converter data link 95. Data resulting from the conversion of the analog signals in the analog-to-digital converter 81 is utilized by the waveform analysis and correlation unit 94 using shared memory (not shown), with the results being delivered to the main computer CPU 90 by waveform data link 96.

Again referring to FIG. 3, communication between the micro-controller board CPU 60 and the main computer CPU 90 is sent over data link 49. In a preferred embodiment, the micro-controller board 46 is positioned inside the main computer 47, and data is transferred by utilizing shared memory and a PCI bus (not shown).

Referring to FIG. 3, the ejector timing circuit 64, previously disclosed, receives an input 61 from the micro-controller CPU 60 for synchronization purposes. The ejector timing circuit 64 is also electrically coupled to the main computer CPU 90 by the ejector timing link 97, through which it receives data input from the main computer CPU 90. The ejector timing circuit 64, and the product ejector 20, are also electrically coupled together by the product ejector link 98. The resulting commands from the ejector timing circuit 64 are also sent to the product ejector 20 by the product ejector link 98.

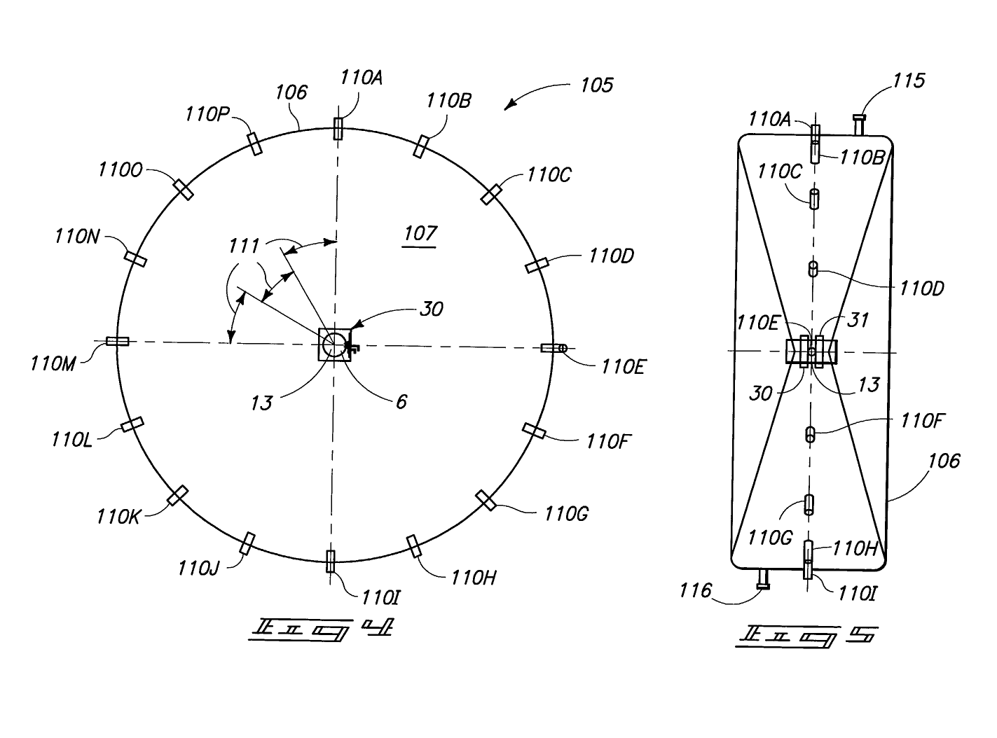

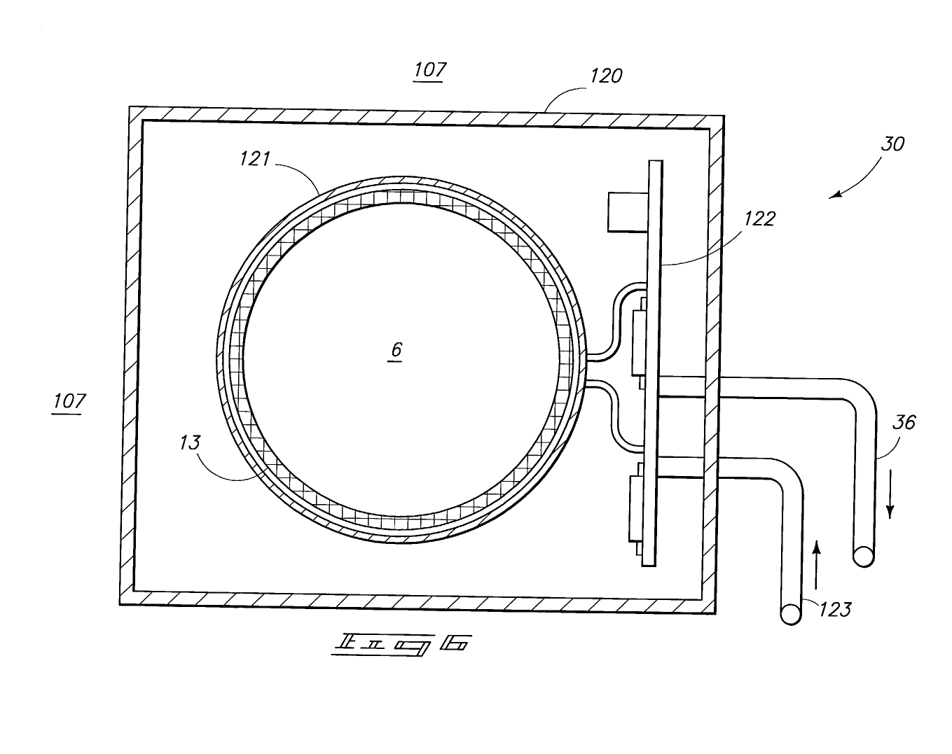

Now referring to FIG. 4, a diagrammatic, fragmentary, transverse sectional view of a portion of the inspection station is generally indicated by the numeral 105. As shown, the inspection station includes a detection chamber housing 106 which is filled with an acoustic medium 107. The acoustic medium 107 is chosen to provide low acoustic attenuation and electrical insulation. An example of such a medium is deionized water with an attenuation coefficient of about 0.042 dB/inch. The detection chamber housing 106 also functions as an electrical field shield which reduces the influence of external electric fields on the first and second electromagnetic sensors 30 and 31 (FIG. 5). Only the first electromagnetic sensor 30 is visible in FIG. 4, while both the first and second electromagnetic sensors 30 and 31 are shown in FIG. 5. The differentiation section 13 is shown to pass through the approximate center of the detection chamber housing 106. In a preferred embodiment, the detection chamber housing 106 is fabricated with 16 gauge welded stainless steel. The housing has an outer diameter of about 48 inches. As best seen by a study of FIGS. 4 and 5, the depth of the detection chamber housing 106 is about 15 inches at the perimeter, and narrows to about 4.75 inches at the approximate center of same.

As seen in FIG. 4, sixteen ultrasonic transducers labeled 110A-P are located in a substantially radially disposed, equally spaced relation about the outer perimeter of the detection chamber housing 106. These sixteen ultrasonic transducers 110A-P are also radially positioned relative to the differentiation section 13 which is located at the approximate center of the detection chamber housing 106. By positioning these same sixteen ultrasonic transducers 110A-P in this orientation, the acoustic energy generated by the respective ultrasonic transducers is directed toward the plurality of articles in the product stream 6 from a number of predetermined angles. Four of these predetermined angles are indicated by the numeral 111. However, as should be understood from a study of FIG. 4, each of the sixteen ultrasonic transducers 110A-P will direct the acoustic energy toward the product stream 6 from a different predetermined angle. Although, as illustrated, sixteen ultrasonic transducers 110A-P are employed, other configurations using varying numbers of ultrasonic transducers are conceivable. For example, as few as one, or as many as one thousand ultrasonic transducers my be practical in some applications depending on the ratio of the electro-mechanical conversion rates of the acceptable and the non-acceptable articles in the product stream. The number of ultrasonic transducers utilized in a given embodiment represents a balance between the complexity and detection capability of the apparatus.

As should be understood, the radial placement of the sixteen ultrasonic transducers 110A-P relative to the product stream 6, allows the acoustic energy generated by same to be directed at the articles in the product stream 6 from a plurality of angles 111 from which they may be subsequently selectively sampled. Therefore, any influence which the orientation of the articles within the product stream 6 may have on the analysis is minimized. This provides for more consistent results. Yet further, it has been discovered that utilizing a plurality of angles 111 also serves to decrease any differences created by the directionality of electromechanical conversion properties of each individual article in the product stream 6.

Referring to FIG. 4, the inner surface or face of each of the ultrasonic transducers 110A-P are located at about twenty four inches from the center of the differentiation section 13. The relatively close proximity of the ultrasonic transducers 110A-P to the differentiation section 13 minimizes the length of the acoustic path over which the acoustic energy must travel, thus substantially preserving the amplitude of the acoustic signal. Commercially acceptable ultrasonic transducers 110A-P are available from Stavely NDT Technologies as undamped 2.25 MHz, ¾ inch immersion ultrasonic transducers (model number 17-0212-P), these transducers have a 6 dB beam width of plus or minus 1.5 degrees.

The differentiation section 13 is preferably constructed of substantially acoustically transmissive material which minimizes attenuation of the acoustic energy while simultaneously possessing low electromechanical conversion properties. The material polyethylene is ideally suited to this application because its inherent molecular charge symmetry makes it an inefficient converter of mechanical to electrical energy. In a preferred embodiment, the material utilized to construct the differentiation section is a stress relieved polyethylene having a 1/16 inch thickness, although other materials with similar characteristics may be used with equal success.

Referring now to FIG. 5, a side elevation view of the detection chamber housing 106 is shown. As seen, a fill tube 115 is provided for introducing new acoustic medium into the detection chamber housing 106. The acoustic medium may also be drained from the detection chamber housing 106 through a drain tube 116 which is provided.

Still referring to FIG. 5, the first and second electromagnetic sensors 30 and 31 are positioned immediately adjacent to, and on opposing sides of the differentiation section 13, and substantially outside the acoustic path. In this regard, the first and second electromagnetic sensors 30 and 31 provide a redundancy of signals, and this correspondingly increases the signal to noise ratio. As noted earlier, other embodiments could conceivably have as few as one sensor depending on the application.

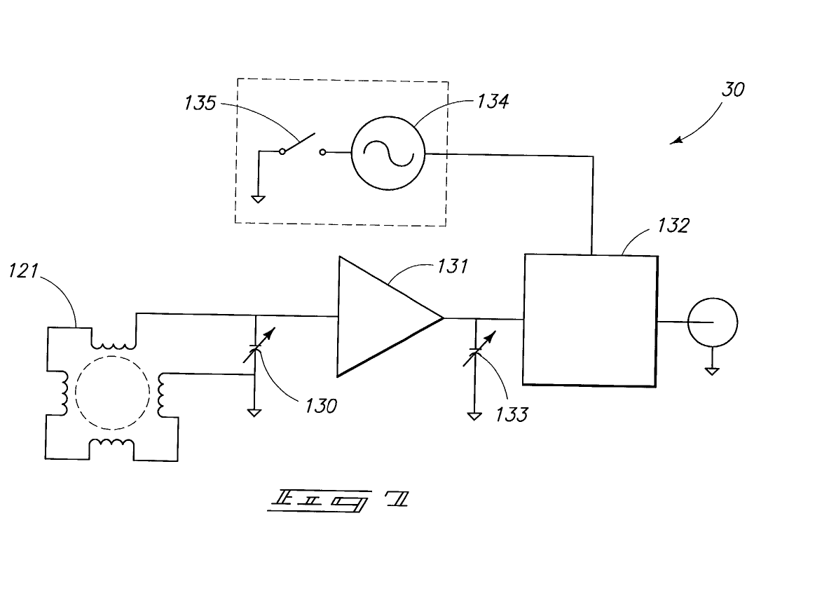

Referring now to FIGS. 5 and 6, a sectional view illustrating the first electromagnetic sensor 30 is shown (FIG. 6). The second electromagnetic sensor 31 is of similar construction, but as best seen in FIG. 5, the first and second electromagnetic sensors 30 and 31 are positioned on the opposite sides of the differentiation section 13. The description of the first electromagnetic sensor 30 which follows applies equally to the second electromagnetic sensors 31.

The electromagnetic sensor housing 120 shields the enclosed circuitry from the acoustic medium 107, and further provides attenuation of any external electric fields which may be present. In a preferred embodiment, the electromagnetic sensor housing 120 is constructed of 18 gauge stainless steel. Further, the dimensions of the electromagnetic sensor housing 120 are approximately 3½ inches by 3 inches, with a thickness of about 1 inch.

As shown in FIG. 6, positioned within the electromagnetic sensor housing 120 is a solenoid coil 121. The electromagnetic signals resulting from the electro-mechanical conversion of the acoustic signal are captured by the solenoid coil 121. In a preferred embodiment, this solenoid coil 121 is composed of about 25 turns of 24 gauge wire, with the turns wound coincident to each other, and further wound over the differentiation section 13. Signals captured by the solenoid coil 121 are amplified and mixed using circuitry contained on a low noise amplifier and mixer circuit board 122.

Referring now to FIG. 6, control signals for timing and calibration as well as power are delivered to the low noise amplifier and mixer circuit board 122 of the first electromagnetic sensor 30 by the first electromagnetic sensor control cable or pathway 123. Similarly, control signals for timing and calibration as well as power are delivered to the low noise amplifier and mixer circuit board 122 of the second electromagnetic sensor (not shown) by the second electromagnetic sensor control cable or pathway (not shown). In the present design, care must be taken to bypass all of the wires in these cables in order to prevent unwanted interference from entering the circuitry. In a preferred embodiment this is accomplished using shielded cables and feed-through capacitors.

Referring to FIGS. 3 and 6, the output signal from the mixer circuit board 122 of the first electromagnetic sensor 30 is sent to the first signal processor board 35 by way of the first signal path or coax 36. Similarly, the output signal from the mixer circuit board 122 of the second electromagnetic sensor 31 is sent to the second signal processor board 37 by a second signal coax or pathway 38. In a preferred embodiment, these respective pathways are impedance matched cables.

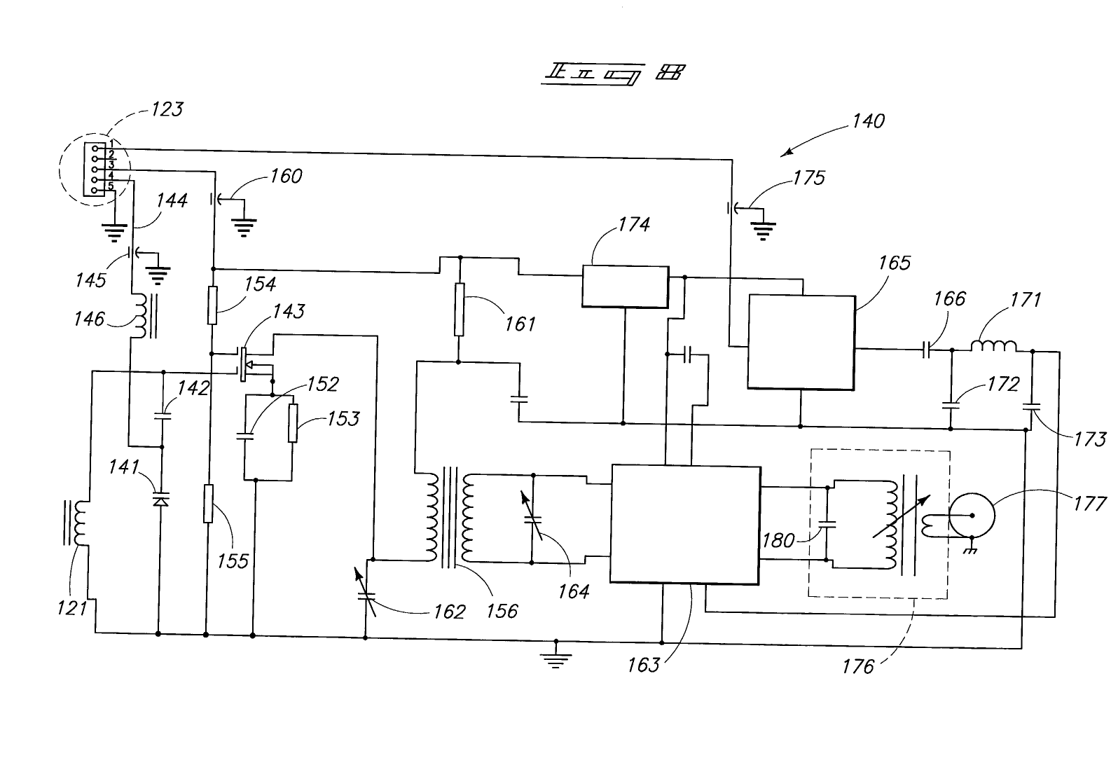

Referring now to FIG. 7, a block diagram representing the first electromagnetic sensor is generally indicated by the numeral 30. The second electromagnetic sensor (not shown) is of similar construction, and the description provided below applies equally well to the second electromagnetic sensor.

As shown in FIG. 7, the solenoid coil 121 forms a parallel resonant circuit with a gate tuning circuit 130. The resonant frequency of this combination should match the frequency of the acoustic energy, and therefore should also match the frequency of the converted electromagnetic energy. This signal is amplified by a low noise amplifier 131 with a gain appropriate to overcome the noise of a mixer 132. A drain tuning circuit 133 is employed to improve gain and signal to noise performance. An up-conversion technique is used to prevent transient overload of the downstream circuitry from energy used to excite the ultrasonic transducers (not shown). For purposes of this application, up-conversion is a process whereby a first signal having a predetermined frequency is combined or mixed with a second signal having a second frequency to provide an output with a different or changed frequency. A mixer local oscillator switch 135 is provided and which triggers a burst from a mixer local oscillator 134. This serves to combine with the incoming signal through the mixer 132 to form the output frequency. In a preferred embodiment, the burst frequency is 2.25 MHz, the local oscillator frequency is 12.95 MHz. The subsequent output frequency is 10.7 MHz.

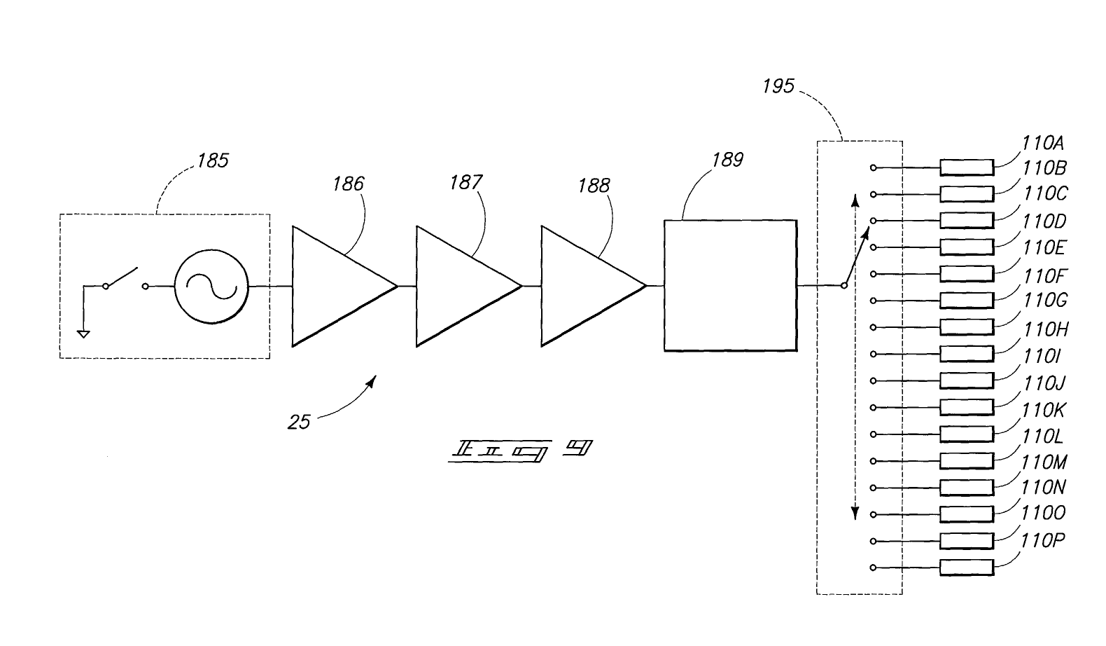

Now turning to FIG. 8, a schematic representation of the first electromagnetic sensor 30 is generally indicated by the numeral 140. The second electromagnetic sensor (not shown) is of similar construction, and the description provided below applies equally well to the second electromagnetic sensor. Emitted electromagnetic signals from the products in the product stream induce voltage in the solenoid coil 121. A parallel resonant circuit is formed with the combination of a tuning diode 141; a tuning coupling capacitor 142 having a nominal value of about 0.01 uF; and the solenoid coil 121 which provides a high impedance circuit which is connected to an appropriate gate of the dual-gate MOSFET 143. In the arrangement shown, the MOSFET is an NTE-455 transistor, although other high gain, low noise N-channel dual-gate MOSFETs may also be used with equal success assuming they are appropriately biased.

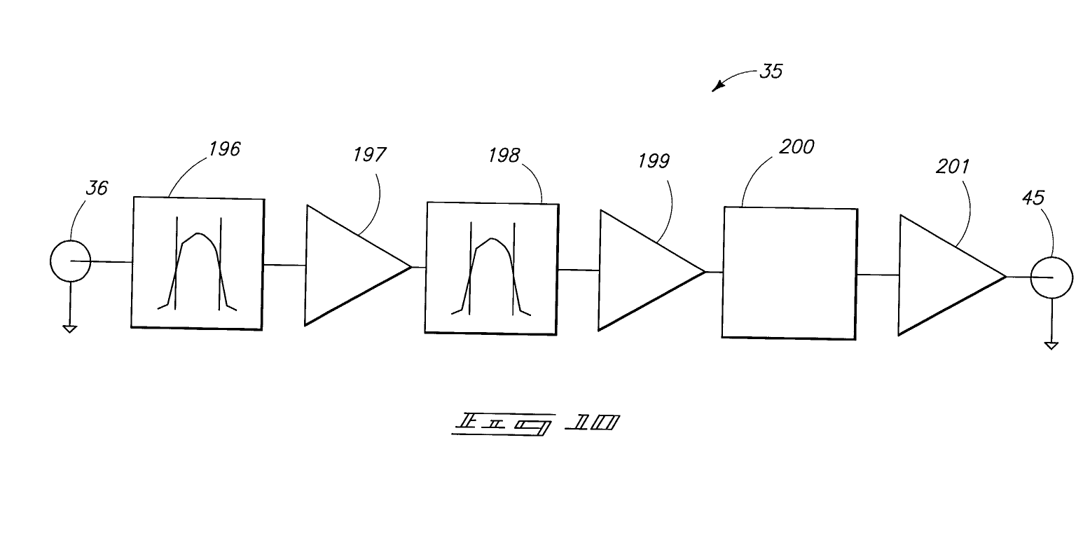

The maximum signal response for the circuit shown in FIG. 8 should coincide with the burst frequency of the burst generator (shown in FIG. 9) which in this invention is about 2.25 MHz. Remote adjustment of the tuning diode 141 is necessary due to its physical location in the arrangement illustrated. In this embodiment, a Crystalonics MV1405 hyper-abrupt varactor diode with a maximum capacitance of about 300 pF accomplishes this purpose. The tuning voltage is delivered by a gate calibration link 144 which is contained within the first electromagnetic sensor control cable or pathway 123. This voltage is bypassed through a capacitor 145 having a value of about 0.1 uF, and a blocking inductor 146 which has a value of about 100 uH. This arrangement shunts high frequency interference.

The resulting signal is amplified using the dual-gate MOSFET 143 in a common source mode, with the source connected to a source AC coupling capacitor 152 having a value of about 0.01 uF; and a source resistor 153 having a value of about 50 ohms connected to common. Bias for gate-two of the MOSFET 143 is provided by a midpoint tap of the series connected gate-two bias resistor 154 connected to 12 Volts DC and a gate-two bias resistor 155 connected to common. These resistors have values of about 100,000 ohms.

The drain of the dual gate MOSFET 143 is coupled through the primary side of a signal coupling transformer 156 to a drain feed resistor 161 having a value of about 100 ohms and which is further connected to 12 Volts DC. This voltage is delivered through an electrical pathway provided within the electromagnetic sensor control cable or pathway 123 and bypasses through a feed through capacitor 160. The signal coupling transformer 156 has a primary and secondary inductance of about 34 uH.

The drain circuit discussed, immediately above, is impedance matched through a parallel resonance circuit formed by the combination of the primary side of signal coupling transformer 156, and the drain tuning capacitor 162. In this embodiment, this variable capacitor 162 has a maximum value of about 300 pF.

The single ended amplified signal in the primary of the signal coupling transformer 156 is provided to the secondary of the signal coupling transformer 156. This arrangement provides isolation and differential input for the mixer 163, which in this embodiment is secured from Phillips Electronics as a Phillips SA-602A; or Signetics NE-602 double-balanced mixer and oscillator.

A parallel resonant circuit is formed between the secondary of signal coupling transformer 156; and input tuning capacitor 164. The tuning capacitor 164 has a maximum value of 470 pF. The secondary of the signal coupling transformer 156 is impedance matched to the mixer 163. In a preferred embodiment, tuning capacitor 164 should be adjusted for maximum signal response at the burst frequency of about 2.25 MHz.

Excitation of the local oscillation input is provided by the combination of a local oscillator 165, and the low pass pi-network filter formed by capacitor 166 which has a value of about 270 pF; a low pass filter inductor 171 having a value of about 1.30 uH secured commercially from Toko as model TK4203, and first and second low pass filter capacitors 172 and 173 which each have values of about 270 pF. The local oscillator 165 is powered by way of a voltage regulator 174 which delivers a regulated 5 Volts DC. This is an industry standard 7805 three terminal regulator which is available from many commercial sources.

Local oscillator 165 is enabled by a signal which travels along a signal path provided with the electromagnetic sensor pathway 123. The signal is bypassed by capacitor 175. In the present embodiment, the local oscillator 165 is secured from Epson Electronics as model SG-8002DC-PTC-ND. It has a frequency of about 12.95 MHz.

Desired signal output from mixer 163 is approximately centered at a frequency of about 10.7 MHz. This signal output is coupled to the primary of output transformer 176 with an integral parallel resonant capacitor 180. The secondary of output transformer 176 is connected to the signal coax pathway 177. In this embodiment, output transformer 176 is a commercially available Toko TK1238.

Referring now to FIG. 9, a block diagram illustrating the acoustic generator 25 is shown. The acoustic signal is created by employing a burst generator 185 that is controlled by a transducer selection circuit 63 circuit which is shown in detail in FIG. 3. Returning to FIG. 9, the burst energy provided by the burst generator is centered at the burst frequency, which in this invention is about 2.25 MHz. In view of the fact that this signal is low in amplitude, and high in impedance it must be amplified before it is capable of driving the ultrasonic transducers 110A-P. This amplification is accomplished by feeding the burst energy to a burst pre-amplifier 186, and then subsequently to a burst intermediate amplifier 187, and then followed by a burst power amplifier 188. These amplifiers may be class A, AB, B or C. The maximum power transfer is achieved by providing a conjugate impedance match relative to the ultrasonic transducers 110A-P. This is accomplished by means of an impedance matcher 189.

In the preferred embodiment, the peak voltage delivered to the respective ultrasonic transducers 110A-P must be less than about 1000 volts (peak to peak) to maintain the maximum lifetime of the ultrasonic transducers. In a preferred embodiment the following commercially available components are utilized. The burst generator utilized is a commercially available Stanford Research Systems model #DS345 synthesizer. The burst pre-amplifier which is employed is a Ramsey Electronics, Inc. LPA-1HF. The burst intermediate amplifier which is employed is a class A, AB, B or C HF linear amplifier with 100 watts output. The burst power amplifier 188 which is employed is a commercially available Ten-Tech model Titan II 1.5 kW HF Amp. The impedance matcher 189 which is employed is a Ten-Tech model #238A L-network antenna tuner capable of handling up to about 2000 watts. Duty cycle for these components is quite low and in a preferred embodiment is about 0.5 percent.

Still referring to FIG. 9, power is provided sequentially to each ultrasonic transducer 110A-P for only brief periods. In this arrangement one transducer is powered while the remaining ones are de-energized. This is accomplished by a power multiplexing circuit 195 which is composed of solid state switches that are selectively controlled by the transducer selection circuit 63 which is shown in FIG. 3. In this way, the electrical power is routed to each ultrasonic transducer 110A-P to focus on a specific area of the differentiation section 13 from a plurality of predetermined angles. In the invention, PIN diode switches and inductors are configured in an arrangement similar to that described and shown in the Microsemi PIN Diode Handbook, Chapter seven, FIG. 7.1. The contents of this reference is incorporated by reference herein.

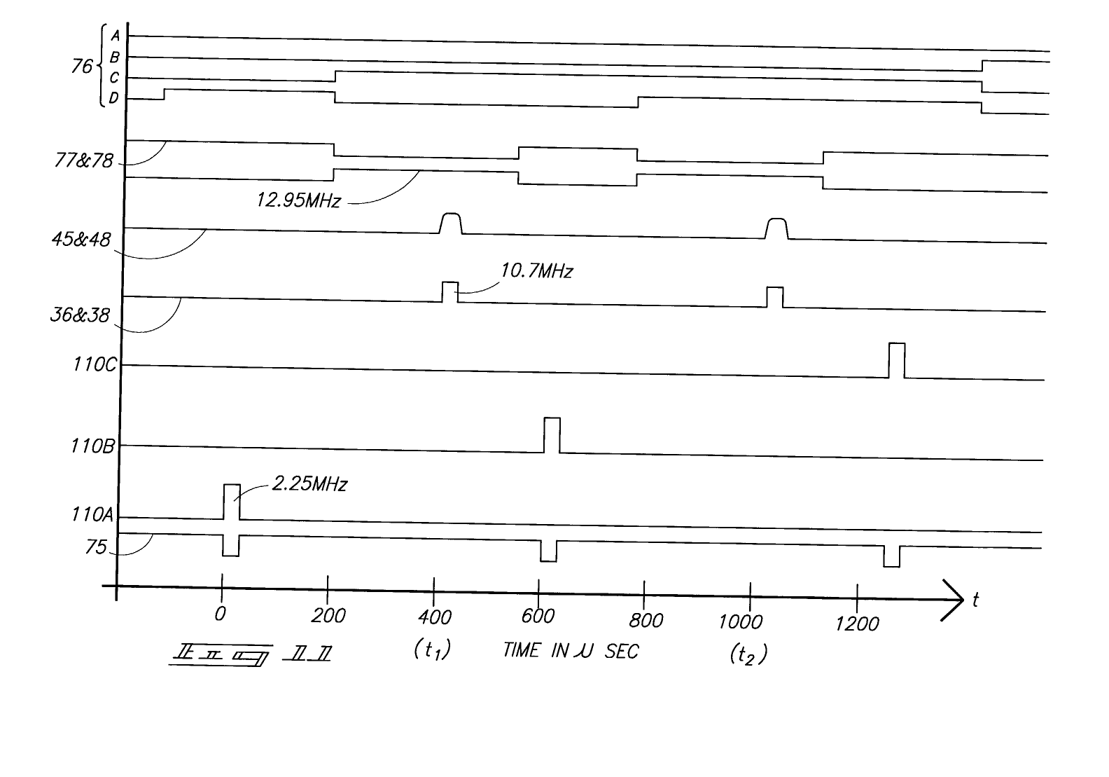

Referring now to FIG. 10, a block diagram showing the signal processor 35 is provided. The electrical signal from the first electromagnetic sensor 30 (FIGS. 2 and 3) is sent over link 36 to a first bandpass filter 196. After the first bandpass filter 196 acts on the signal, it proceeds to the first signal amplifier shown at 197. The filter bandwidth which is selected for the first bandpass filter must be narrow enough to prevent amplifier overload, and wide enough to prevent signal distortion. As shown herein, the center frequency is about 10.7 MHz, with a bandwidth of about 25 KHz. The amplifier exhibits low noise, and has a gain of about 20 dB. Such filters and amplifiers are well known in the art.

The amplified signal is then sent from the first signal amplifier 197 to a second bandpass filter 198. The signal then proceeds to a second signal amplifier 199. Once again, the filter bandwidth of the second bandpass filter must be narrow enough to prevent amplifier overload, and wide enough to prevent significant unwanted signal distortion. In the present invention, the center frequency is about 10.7 MHz with a bandwidth of about 25 KHz. The amplifier which is employed exhibits low noise, and has a gain of about 20 dB. Such filters and amplifiers are well known in the art.

The output of the second signal amplifier 199 is coupled in signal transmitting relation to an input of a demodulator 200. This device recovers the envelope of the original signal. This signal is then amplified by the demodulator amplifier 201. Here the signal is amplified to a level suitable for A/D conversion. In a preferred embodiment, the center frequency of this circuit is about 10.7 MHz with a bandwidth of about 25 Khz.

Still referring to FIG. 10, the signal is rectified in the demodulator 200 and further amplified using the demodulator amplifier 201. The resulting signal voltage level is now high enough to be transmitted by the first processed signal path 45 to the analog-to-digital converter 81. The signal from the second electromagnetic sensor 31 is processed in the same fashion, and is then sent by the second processed signal path 48 to the analog-to-digital converter 81 (shown in FIG. 3).

As discussed above, a product stream containing acceptable and unacceptable articles 8 and 9 is transported to the inspection station 12 where articles 7 in the product stream 6 are differentiated as acceptable and unacceptable by comparing the electromechanical conversion properties of the articles 7 against a reference set by the operator.

As earlier disclosed, the product stream 6 flows through the transport pipe 3, and into the inspection station 12 and arrives at the differentiation section 13. The portion of the product stream within the differentiation section 13 is stimulated by bursts of acoustic energy 26 generated by the acoustic generator 25. As this acoustic stimulation occurs, articles 7 in the product stream 6 convert the received acoustic energy into electromagnetic energy 28 which is emitted at a rate which is related to their respective electro-mechanical conversion properties, and at a frequency substantially equal to that of the acoustic energy which has been received.

For example, it is known in the art that bone and cartilage possess piezoelectric characteristics, that is, they produce electrical potential in response to mechanical pressure. It is also known in the art that muscle and fat exhibit exceedingly small piezoelectric characteristics. In view of this response, as acoustic stimulation occurs, any bone or cartilage in the product stream will convert the mechanical (acoustic) energy to electromagnetic energy at a much higher rate than does the surrounding muscle and fat.

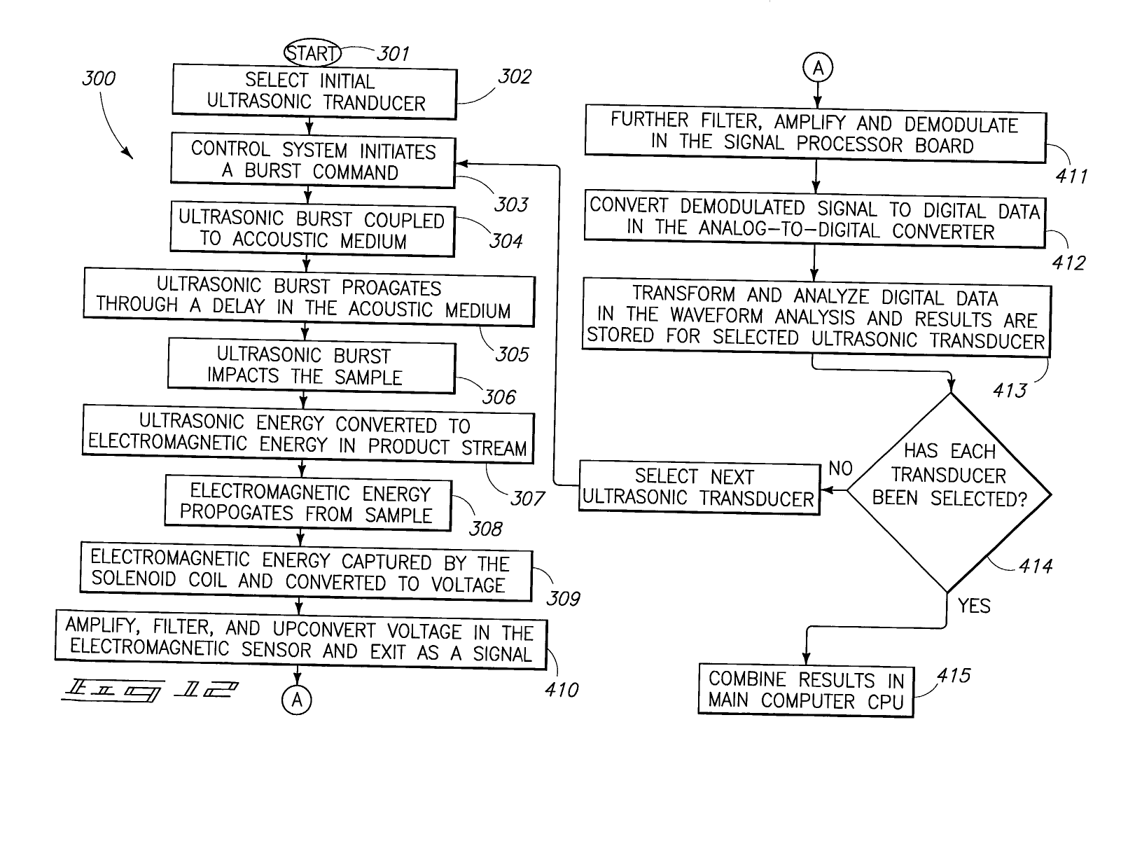

Referring now to FIGS. 1-12, a brief overview of a sampling cycle 300 is discussed. Operation begins when the operator chooses to begin sorting 301 by selecting the appropriate command at the operator interface 91. This information is sent to the main computer CPU 90 and relayed to the micro-controller CPU 60. An inspection cycle is initiated by micro-controller CPU 60 which selects the first ultrasonic transducer 302 (110A) by commanding the transducer selection circuit 63 to begin at h‘0001’. This command is sent by the transducer selector link 76 to the power multiplexing circuit 195 where the power path is routed to send power to ultrasonic transducer 110A.

Next, a burst command 303 is sent to the burst timing circuitry 62. The burst enable link 75 carries a negative logic pulse with a 40 usec duration to the acoustic generator 25, where it instructs the burst generator 185 to oscillate at about 2.25 MHz for 40 usec. This signal is low in amplitude and high in impedance, to generate electrical energy. The electrical energy produced by the burst generator 185 is first amplified by burst pre-amplifier 186, then by the burst intermediate amplifier 187, and finally by the burst power amplifier 188. The impedance matcher 189 then provides a conjugate impedance match to the power multiplexing circuit 195 and ultrasonic transducer 110A.

This burst of electrical energy is then converted to a burst of acoustic or ultrasonic energy by the ultrasonic transducer 110A, and directed toward the differentiation section 13. The acoustic energy travels from the ultrasonic transducer 110A to the differentiation section 13 through the acoustic medium 27. The ultrasonic transducer 110A; the differentiation section 13; and the acoustic medium 27 are acoustically coupled 304 to facilitate transmission of the acoustic energy 26. The burst of acoustic energy 26 travels at a rate of about 4,950 feet per second and reaches the differentiation section 13 in about 400 usec. This speed of travel of the acoustic wave represents a time delay 305. During this time delay, the first and second mixer enable links 77 and 78 are held high (negative logic) prohibiting the mixer 132 from passing residual burst energy to the first and second signal processing boards 35 and 37.

After the burst of acoustic energy 26 has been generated and while the acoustic energy 26 is traveling towards the differentiation section 13, the micro-controller CPU 60 commands the mixer timing circuit 67 to enable the mixer local oscillator 134 by sending a negative logic pulse through the first or second mixer enable links 77 and 78. This occurs about 200 usec after the falling edge of the burst enable link 75 and lasts for about 300 usec. This generates a 300 usec about 12.95 MHz burst which is fed to the local oscillator input of mixer 132. When enabled, this mixing circuit 132 and sequence transforms the input energy from 2.25 MHz to 10.7 MHz.

When the burst of acoustic energy 26 arrives at the differentiation section 13, at least a portion of the acoustic energy 26 is received by articles in the product stream 306 which flows through the differentiation section 13. At least some of the acoustic energy received by the articles is converted into electromagnetic energy 28 by the articles 307. At least some of the electromagnetic energy 28 generated by the conversion 308 is received by the solenoid coil 121 of the first or second electromagnetic sensors 30 and 31 and converted to voltage 309.

Now, referring to FIGS. 2 and 8, electromagnetic energy 26 received by the solenoid 121 and coupled into voltage 309 forms a parallel resonant circuit with the combination of the tuning diode 141 and the tuning coupling capacitor 142. This small signal is then amplified using the dual-gate MOSFET 143 in a common source mode, with the source connected to the source AC coupling capacitor 152, and source resistor 153. Bias for gate-two is provided by a midpoint tap of the series connected to the gate-two bias resistor 154 and which is further coupled to 12 Volts DC; and the gate-two bias resistor 155 is connected to common.

The amplified signal is recovered from the drain of the dual gate MOSFET 143 and is coupled through the primary of the signal coupling transformer 156 to the secondary of signal coupling transformer 156, providing isolation and differential input for the mixer 163 which converts the frequency from 2.25 MHz to 10.7 Mhz when local oscillator 165 is enabled which should be the case when a valid signal is present 410.

The signal exiting at step 410 is coupled through the transformer 176 to the feed first signal path 36 and which is further electrically connected to a first bandpass filter 196 as shown in FIG. 10. This first bandpass filter 196 removes extraneous energy that lies outside its bandwidth to prevent saturation of downstream components and to preserve dynamic range. The signal is further amplified by a first signal amplifier 197 seen at step 411 and delivered to a second bandpass filter 198 and followed by a second signal amplifier 199. The output signal is amplified to the point that it can be effectively rectified in the demodulator 200 (seen at step 412) to recover the envelope of the original signal. This envelope is amplified by the demodulator amplifier 201.

Demodulated output signals are sent by the first processed signal path 45 and its companion signal by the second processed signal path 48 to the analog-to-digital converter 81 (seen at step 413) where time multiplexed digital data representing the demodulated signal is created. Timing signals used to frame the data are sent from the analog-to-digital timing circuit 65 and initiated by the micro-controller CPU 60.

Data from the analog-to-digital converter 81 representing the signal from articles in the differentiation section 13 is transferred to the waveform analysis and correlation unit 94. In one aspect of the invention, the data is normalized using look-up table values which are employed to correct the magnitude response obtained during a calibration cycle performed during setup and maintenance and which is not shown.

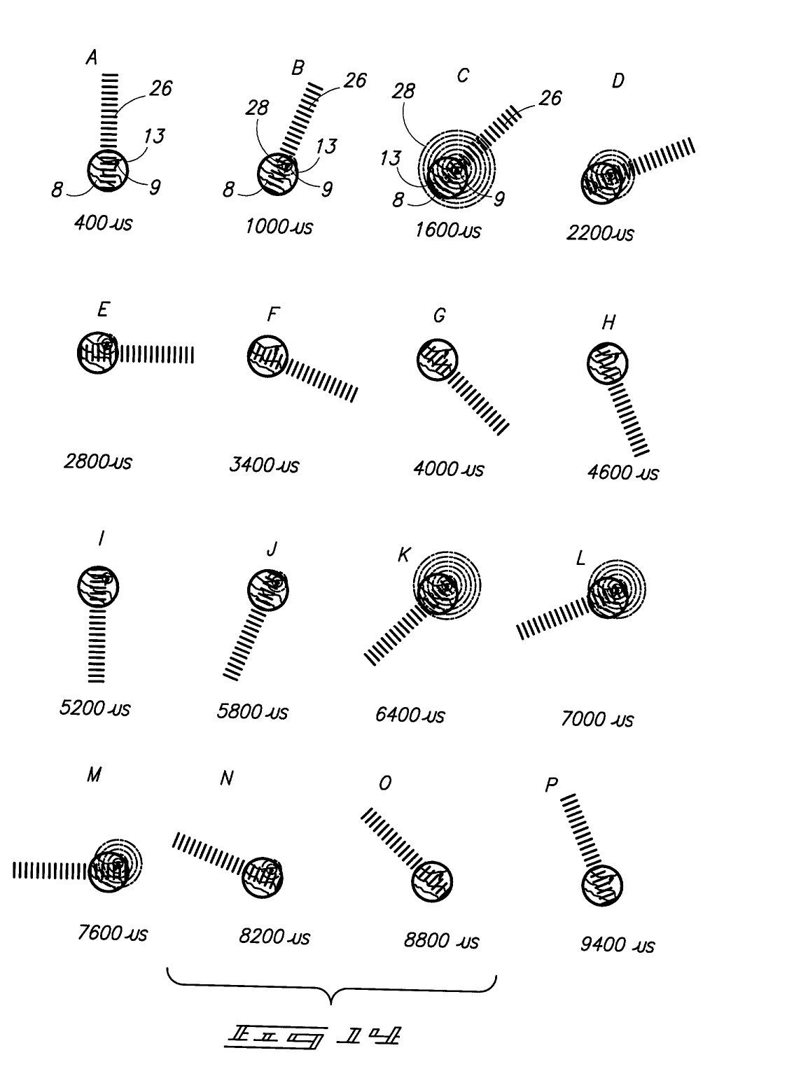

In another aspect of the invention, these signals are further normalized using a lookup table which maps the location of an article of interest in the differentiation section 13 with the magnitude of the signal electrically received. This location is calculated by measuring the time of arrival of the electrical signal and the angular position of the respective ultrasonic transducer 110A. This is utilized as an indicator of the location of the article relative to the boundaries of the differentiation section 13 as illustrated in FIG. 13.

In a preferred embodiment, this normalized data is transferred to the main computer CPU 90 where it is stored temporarily as other orientations of the differentiation section 13 are evaluated. This completes an initial sample from ultrasonic transducer 110A. This process is repeated for each of the transducers 414 and the results are combined in the main computer CPU as seen at step 415.

Referring now to FIG. 13, a diagrammatic representation showing a product stream 6 flowing through the differentiation section 13 is generally indicated by the numeral 209. The product stream 6 includes acceptable articles 8 including meat and unacceptable articles 9 including bone and cartilage having distinguishable electro-mechanical conversion efficiencies. The product stream 6 is shown to be oriented in receiving relation relative to the acoustic energy 26 which is directed toward the product stream 6. An unacceptable article 9 such as bone or cartilage is shown receiving at least some of the acoustic energy 26. Following receipt, the unacceptable article 9 converts at least some of the received acoustic energy 26 into electromagnetic energy 28. As earlier disclosed, a pair of electromagnetic sensors (not shown) are positioned to detect at least some of the electromagnetic energy 28 which is emitted by the conversion of the acoustic energy.

Referring now to FIG. 14, a diagrammatic representation showing a product stream flowing through the differentiation section 13 is shown in sixteen temporally sequential drawings labeled A-P, respectively. Each of the sixteen drawings in the sequence A-P are temporally separated by about 600 usec. In each of these sequential drawings, the product stream includes acceptable articles 8 including meat, and an unacceptable article 9, such as bone or cartilage. As best seen in FIG. 14, the product stream is disposed in receiving relation relative to the acoustic energy 26 which is directed toward the product stream by one of the ultrasonic transducers 110A-P which are radially disposed in a regularly spaced fashion about the outer perimeter of the detection chamber housing 106.

As shown in FIG. 14, an unacceptable article 9, such as bone or cartilage, receives at least some of the acoustic energy 26. As discussed the unacceptable article 9 converts at least some of the received acoustic energy 26 into electromagnetic energy 28. In this regard, the illustrated circles emanating from the unacceptable article 9 represent the electromagnetic energy 28 which is generated by the conversion. In the illustration a greater number of circles represents a higher magnitude of electromagnetic energy 28 emitted; while a smaller number of circles represent a lower magnitude of electromagnetic energy 28 emitted.

As seen in these sequential drawings, the orientation of the acoustic energy 26 relative to the unacceptable article 9 can effect the magnitude of the electromagnetic energy 28 generated. Additionally, the location and orientation of the unacceptable article 9 within the differentiation section 13 can also effect the magnitude of the electromagnetic energy 28 generated.

As illustrated, in sequential drawing A, minimal acoustic energy is received by the unacceptable article 9 due the orientation and location of the unacceptable article 9 within the differentiation section 13. Additionally, minimal electromagnetic energy 28 is created because little acoustic energy 26 was received and converted.

In sequential drawing B, which is about 600 usec later in time, the small increase in the amount of electromagnetic energy 28 generated is due to the fact that more acoustic energy 26 was received by the unacceptable article 9 in view of its more favorable orientation relative to the incoming acoustic energy 26.

In sequential drawing C, which is about 600 usec later in time, a dramatic increase in the electromagnetic energy 28 generated is shown. This is caused by the very favorable orientation of the unacceptable article 9 and the incoming acoustic energy 26.

In remaining sequential drawings D-P the relationship between the orientation of the article and the angle of the incoming acoustic energy 26 is shown as an entire sample cycle of ultrasonic transducers 110D-P is sequentially implemented. A close study of FIG. 14 illustrates how the relationship between the angle of the acoustic energy, and the orientation and the location of the unacceptable article 9 impacts the amount of electromagnetic energy 28 generated by the subsequent conversion process.

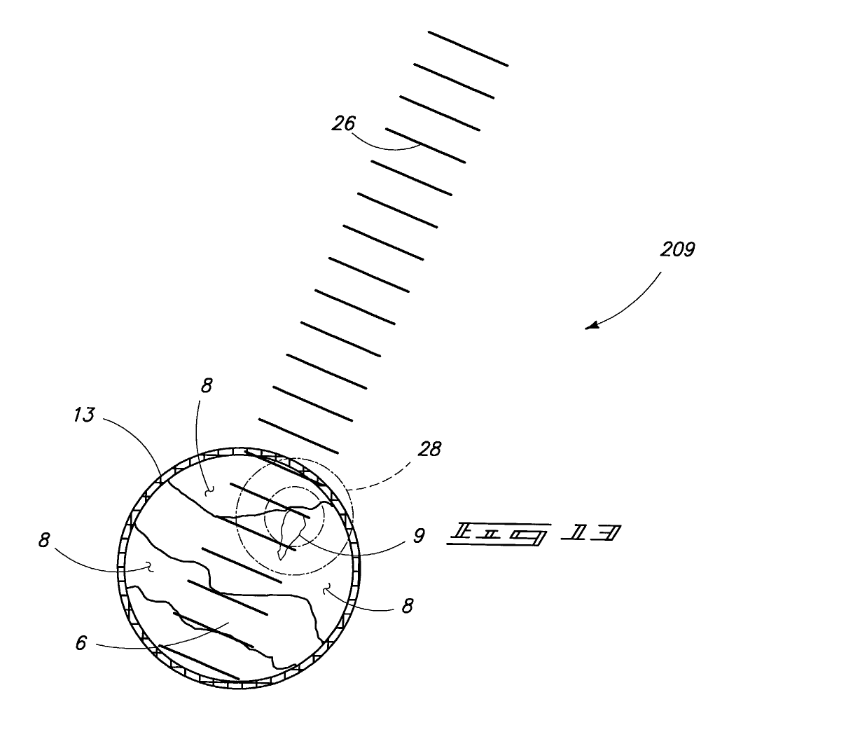

Data from the remaining ultrasonic transducers 100D-P are collected and stored with data from ultrasonic transducer 110A-C in the main computer CPU 90 (step 415 FIG. 12). This data includes both the magnitude of the signal and the time of arrival of same.

This data may be analyzed and utilized in several ways. In one aspect of the invention, the data from each sampled angle is averaged together to form a composite magnitude indicative of the response. In this way, orientation effects are minimized. In another aspect of the invention, the sample data is correlated with location data to form a polar map of the conversion efficiency of the articles in the differentiation section 13. Regions in this polar map are compared against threshold levels set by the user as a means to differentiate the acceptable and unacceptable articles 8 and 9. In a further aspect of the invention, the sample data and location data are further transformed by conversion of polar coordinates to Cartesian coordinates to develop an image representative of the conversion efficiency of the articles 7 in the detection section 13.

As unacceptable articles 9 are identified, a command is sent from the main computer CPU 90 to the ejector timing circuit 64 where it is queued and combined with a synchronization signal transferred by the ejection timing link from the micro-controller CPU 60. This entire cycle is repeated continuously during sorting operations.

After exiting the inspection station 12, the plurality of articles 7 enter the product ejector 20 where acceptable and unacceptable articles 8 and 9 are directed by the product ejector 20 either into the accept pipe 21, or into the reject pipe 22 .

In a second operational mode dedicated to calibration, the operator inserts a calibration apparatus that is composed of a uniform target composed of machined piezoelectric material that has been manufactured to deliver spatially uniform response. This calibration apparatus is centered in a bladder and placed in the differentiation section 13. The operator selects a calibration sequence from the operator interface 91 which is read by the main computer CPU 90 and commands the micro-controller CPU 60 to initiate a sample sequence identical to the cycle outlined earlier.

Operation

The operation of the described embodiment of the present invention is believed to be readily apparent and are briefly summarized at this point.

An apparatus for differentiating articles is disclosed, which includes an acoustic generator 25 for generating acoustic energy 26; a plurality of articles 7 located in receiving relation relative to the acoustic energy 26, and wherein the plurality of articles 7 receive at least a portion of the acoustic energy 26, and wherein at least some of the acoustic energy 26 received by the plurality of articles 7 is converted into electromagnetic energy 28; a sensor 30 and 31 for detecting at least some of the electromagnetic energy 26 generated by the conversion of the acoustic energy 26; and a processor 47 operably coupled with the sensor 30 and 31 for differentiating the plurality of articles 7 based upon the electromagnetic energy sensed.

In addition to the foregoing, an apparatus for differentiating articles is disclosed, which includes an acoustic generator 25 for generating acoustic energy 26; a product stream 6 located in receiving relation relative to the acoustic energy 26, and wherein the product stream 6 includes a plurality of articles 7, and wherein at least some of the acoustic energy 26 is received by the plurality of articles 7, and wherein at least some of the acoustic energy received by the plurality of articles 7 is converted into electromagnetic energy 28; a sensor 30 and 31 for detecting at least some of the electromagnetic energy 28 generated by the conversion; and a processor 47 operably coupled with the sensor 30 and 31 for differentiating the plurality of articles 7.

In addition to the foregoing, an apparatus for differentiating articles is disclosed, which includes a product stream 6 which includes acceptable and unacceptable articles 8 and 9 with differing electromechanical conversion efficiencies; an acoustic generator 25 which produces acoustic energy 26 which is directed to the articles 7 traveling in the product stream 6, and wherein the respective articles based upon their individual electromechanical conversion efficiencies convert the acoustic energy 26 into electromagnetic energy 28; a sensor 30 and 31 for receiving at least some of the electromagnetic energy 28 generated by the conversion of the acoustic energy 26; and a processor 47 operably coupled with the sensor for differentiating between the acceptable and unacceptable articles 8 and 9 based on their respective electromechanical conversion efficiencies.

In addition to the foregoing, an apparatus for differentiating acceptable and unacceptable articles 8 and 9 in a product stream 6 is disclosed, which includes an acoustic generator 25 for generating acoustic energy 26, a product stream 6 located in receiving relation relative to the acoustic energy 26, and which includes acceptable articles 8 including meat and unacceptable articles 9 including bone and cartilage, and wherein the acceptable and unacceptable articles 8 and 9 have differing electro-mechanical conversion efficiencies, and wherein the acceptable and unacceptable articles 8 and 9 receive at least some of the acoustic energy 26, and wherein the acceptable and unacceptable articles 8 and 9 convert at least some of the received acoustic energy into electromagnetic energy 28; a sensor 30 and 31 for detecting at least some of the electromagnetic energy 28 generated by the conversion; and a processor 47 operably coupled with the sensor 30 and 31 for differentiating between the acceptable and unacceptable articles 8 and 9 based on their respective electro-mechanical conversion efficiencies.

In addition to the foregoing, an apparatus for differentiating acceptable and unacceptable articles 8 and 9 in a product stream 6 is disclosed, and which includes an acoustic generator 25 including a plurality of transducers 110A-P positioned at predetermined angles relative to the product stream 6 for generating acoustic energy 26; a product stream 6 located in receiving relation relative to the acoustic energy 26, and wherein the product stream 6 includes acceptable articles 8 consisting of meat and unacceptable articles 9 which include bone and cartilage, and wherein the acceptable and unacceptable articles 8 and 9 have differing electro-mechanical conversion efficiencies, and wherein the acceptable and unacceptable articles 8 and 9 receive at least some of the acoustic energy 26, and wherein the acceptable and unacceptable articles 8 and 9 convert at least some of the received acoustic energy into electromagnetic energy 28; a sensor 30 and 31 for detecting at least some of the electromagnetic energy 28 generated by the conversion of the acoustic energy 26; and a processor 47 operably coupled with the sensor 30 and 31, and which utilizes information received from the sensor 30 and 31 to map the electro-mechanical conversion efficiencies of the acceptable and unacceptable articles 8 and 9, and to differentiate between the acceptable and unacceptable articles 8 and 9.

Still further, the apparatus for differentiating articles includes differentiating a plurality of articles 7 which include food products, and which include meat, bone and cartilage. As disclosed above, the acoustic generator 25 includes at least one transducer. In a preferred embodiment, the acoustic generator 25 includes a plurality of transducers 110A-P. Still further, in a preferred embodiment, the plurality of transducers 110A-P direct the acoustic energy 26 toward a plurality of articles from a plurality of predetermined angles 111. Still further, in a preferred embodiment, the acoustic generator 25 includes a plurality of transducers 110A-P which direct the acoustic energy 26 toward the articles from a plurality of predetermined angles 111, and the processor 47 receives information from the plurality of predetermined angles and averages same. Still further, the sensor 30 and 31 includes a solenoid coil 121. As disclosed above, the acoustic energy 26 has a predetermined frequency, and the electromagnetic energy 28 generated by the conversion has a frequency, and the frequency of the acoustic energy 26 and the frequency of the electromagnetic energy 28 generated by the conversion are substantially equal. Still further, the acoustic generator 25 includes a plurality of transducers 110A-P for generating acoustic energy 26, and the plurality of transducers 110A-P do not generate acoustic energy 26 while the articles convert previously emitted acoustic energy into electromagnetic energy 28. Still further, the processor utilizes the relative conversion efficiencies of the plurality of articles 7 to map their respective locations.

In addition to the foregoing, a method for differentiating articles is disclosed, and which includes generating acoustic energy 303; directing the acoustic energy 26 toward a product stream 305 which includes acceptable and unacceptable articles 8 and 9 having differing electromechanical conversion efficiencies; converting at least some of the acoustic energy received by the acceptable and unacceptable articles 8 and 9 into electromagnetic energy 28 at rates relative to the acceptable and unacceptable articles 8 and 9 respective electromechanical conversion efficiencies 307; detecting at least some of the electromagnetic energy 28 generated by the conversion 309; and differentiating the acceptable and unacceptable articles 8 and 9 based upon their respective electro-mechanical conversion efficiencies as indicated by the sensed electromagnetic energy 415.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

1. An apparatus for differentiating articles, comprising:
an acoustic generator for generating acoustic energy;
a plurality of articles located in receiving relation relative to the acoustic energy, and wherein the plurality of articles receive at least a portion of the acoustic energy, and wherein at least some of the acoustic energy received by the plurality of articles is converted into electromagnetic energy;
a sensor for detecting at least some of the electromagnetic energy generated by the conversion of the acoustic energy; and
a processor operably coupled with the sensor for differentiating the plurality of articles based upon the electromagnetic energy sensed.

2. The apparatus of claim 1, wherein the plurality of articles comprise food products.

3. The apparatus of claim 1, wherein the plurality of articles comprise meat, bone and cartilage.

4. The apparatus of claim 1, wherein the acoustic generator comprises at least one transducer.

5. The apparatus of claim 1, wherein the acoustic generator comprises a plurality of transducers.

6. The apparatus of claim 1, wherein the acoustic generator comprises a plurality of transducers which direct the acoustic energy toward the plurality of articles from a plurality of predetermined angles.

7. The apparatus of claim 1, wherein the acoustic generator comprises a plurality of transducers which direct the acoustic energy toward the articles from a plurality of predetermined angles, and wherein the processor receives information from the plurality of predetermined angles, and wherein the information received from the plurality of predetermined angles is averaged.

8. The apparatus of claim 1, wherein the sensor comprises a solenoid coil.

9. The apparatus of claim 1, wherein the acoustic energy has a predetermined frequency, and wherein the electromagnetic energy generated by the conversion has a frequency, and wherein the frequency of the acoustic energy and the frequency of the electromagnetic energy generated by the conversion are substantially equal.

10. The apparatus of claim 1, wherein the acoustic generator comprises a plurality of transducers for generating acoustic energy, and wherein the plurality of transducers do not generate acoustic energy while the articles convert previously emitted acoustic energy into electromagnetic energy.

11. The apparatus of claim 1, wherein the processor utilizes the relative conversion efficiencies of the plurality of articles to map their respective locations.

12. An apparatus for differentiating articles in a product stream, comprising:
an acoustic generator for generating acoustic energy;
a product stream located in receiving relation relative to the acoustic energy, and wherein the product stream includes a plurality of articles, and wherein at least some of the acoustic energy is received by the plurality of articles, and wherein at least some of the acoustic energy received by the plurality of articles is converted into electromagnetic energy;
a sensor for detecting at least some of the electromagnetic energy generated by the conversion; and
a processor operably coupled with the sensor for differentiating the plurality of articles.

13. The apparatus of claim 12, wherein the plurality of articles comprise food products.

14. The apparatus of claim 12, wherein the plurality of articles comprise meat, bone and cartilage.

15. The apparatus of claim 12, wherein the acoustic generator comprises at least one transducer.

16. The apparatus of claim 12, wherein the acoustic generator comprises a plurality of transducers.

17. The apparatus of claim 12, wherein the acoustic generator comprises a plurality of transducers which direct the acoustic energy toward the plurality of articles from a plurality of predetermined angles.

18. The apparatus of claim 12, wherein the acoustic generator comprises a plurality of transducers which direct the acoustic energy toward the articles from a plurality of predetermined angles, and wherein the processor receives information from the plurality of predetermined angles, and wherein the information received from the plurality of predetermined angles is averaged.

19. The apparatus of claim 12, wherein the sensor comprises a solenoid coil.

20. The apparatus of claim 12, wherein the acoustic energy has a predetermined frequency, and wherein the electromagnetic energy generated by the conversion has a frequency, and wherein the frequency of the acoustic energy and the frequency of the electromagnetic energy generated by the conversion are substantially equal.

21. The apparatus of claim 12, wherein the acoustic generator comprises a plurality of transducers for generating acoustic energy, and wherein the plurality of transducers do not generate acoustic energy while the articles convert previously emitted acoustic energy into electromagnetic energy.

22. The apparatus of claim 12, wherein the processor utilizes the relative conversion efficiencies of the plurality of articles to map their respective locations.

23. An apparatus for differentiating articles in a product stream, comprising:
a product stream which includes acceptable and unacceptable articles with differing electro-mechanical conversion efficiencies;
an acoustic generator which produces acoustic energy which is directed to the articles traveling in the product stream, and wherein the respective articles based upon their individual electromechanical conversion efficiencies convert the acoustic energy into electromagnetic energy;
a sensor for receiving at least some of the electromagnetic energy generated by the conversion of the acoustic energy; and
a processor operably coupled with the sensor for differentiating between the acceptable and unacceptable articles based on their respective electromechanical conversion efficiencies.

24. The apparatus of claim 23, wherein the acceptable and unacceptable articles comprise food products.

25. The apparatus of claim 23, wherein the acceptable and unacceptable articles comprise meat, bone and cartilage.

26. The apparatus of claim 23, wherein the acoustic generator comprises at least one transducer.

27. The apparatus of claim 23, wherein the acoustic generator comprises a plurality of transducers.

28. The apparatus of claim 23, wherein the acoustic generator comprises a plurality of transducers which direct the acoustic energy toward the plurality of articles from a plurality of predetermined angles.

29. The apparatus of claim 23, wherein the acoustic generator comprises a plurality of transducers which direct the acoustic energy toward the acceptable and unacceptable articles from a plurality of predetermined angles, and wherein the processor receives information from the plurality of predetermined angles, and wherein the information received from the plurality of predetermined angles is averaged.

30. The apparatus of claim 23, wherein the sensor comprises a solenoid coil.

31. The apparatus of claim 23, wherein the acoustic energy has a predetermined frequency, and wherein the electromagnetic energy generated by the conversion has a frequency, and wherein the frequency of the acoustic energy and the frequency of the electromagnetic energy generated by the conversion are substantially equal.

32. The apparatus of claim 23, wherein the acoustic generator comprises a plurality of transducers for generating acoustic energy, and wherein the plurality of transducers do not generate acoustic energy while the articles convert previously emitted acoustic energy into electromagnetic energy.

33. The apparatus of claim 23, wherein the processor utilizes the relative conversion efficiencies of the acceptable and unacceptable articles to map their respective locations.

34. An apparatus for differentiating acceptable and unacceptable articles in a product stream, comprising:
an acoustic generator for generating acoustic energy
a product stream located in receiving relation relative to the acoustic energy, and which includes acceptable articles including meat and unacceptable articles including bone and cartilage, and wherein the acceptable and unacceptable articles have differing electro-mechanical conversion efficiencies, and wherein the acceptable and unacceptable articles receive at least some of the acoustic energy, and wherein the acceptable and unacceptable articles convert at least some of the received acoustic energy into electromagnetic energy;
a sensor for detecting at least some of the electromagnetic energy generated by the conversion; and
a processor operably coupled with the sensor for differentiating between the acceptable and unacceptable articles based on their respective electro-mechanical conversion efficiencies.

35. The apparatus of claim 34, wherein the acoustic generator comprises at least one transducer.

36. The apparatus of claim 34, wherein the acoustic generator comprises a plurality of transducers.

37. The apparatus of claim 34, wherein the acoustic generator comprises a plurality of transducers which direct the acoustic energy toward the plurality of articles from a plurality of predetermined angles.

38. The apparatus of claim 34, wherein the acoustic generator comprises a plurality of transducers which direct the acoustic energy toward the acceptable and unacceptable articles from a plurality of predetermined angles, and wherein the processor receives information from the plurality of predetermined angles, and wherein the information received from the plurality of predetermined angles is averaged.

39. The apparatus of claim 34, wherein the acoustic energy has a predetermined frequency, and wherein the electromagnetic energy generated by the conversion has a frequency, and wherein the frequency of the acoustic energy and the frequency of the electromagnetic energy generated by the conversion are substantially equal.

43. The apparatus of claim 34, wherein the acoustic generator comprises a plurality of transducers for generating acoustic energy, and wherein the plurality of transducers do not generate acoustic energy while the articles convert previously emitted acoustic energy into electromagnetic energy.

44. The apparatus of claim 34, wherein the processor utilizes the relative conversion efficiencies of the acceptable and unacceptable articles to map their respective locations.

45. An apparatus for differentiating acceptable and unacceptable articles in a product stream, comprising:
an acoustic generator including a plurality of transducers positioned at predetermined angles relative to the product stream for generating acoustic energy;
a product stream located in receiving relation relative to the acoustic energy, and wherein the product stream includes acceptable articles consisting of meat and unacceptable articles which include bone and cartilage, and wherein the acceptable and unacceptable articles have differing electro-mechanical conversion efficiencies, and wherein the acceptable and unacceptable articles receive at least some of the acoustic energy, and wherein the acceptable and unacceptable articles convert at least some of the received acoustic energy into electromagnetic energy;
a sensor for detecting at least some of the electromagnetic energy generated by the conversion of the acoustic energy; and
a processor operably coupled with the sensor, and which utilizes information received from the sensor to map the electro-mechanical conversion efficiencies of the acceptable and unacceptable articles, and to differentiate between the acceptable and unacceptable articles.

46. The apparatus of claim 45, wherein the processor receives information from the plurality of transducers positioned at predetermined angles, and wherein the information received from the plurality of predetermined angles is averaged.

47. The apparatus of claim 45, wherein the sensor comprises a solenoid coil.

48. The apparatus of claim 45, wherein the acoustic energy has a predetermined frequency, and wherein the electromagnetic energy generated by the conversion has a frequency, and wherein the frequency of the acoustic energy and the frequency of the electromagnetic energy generated by the conversion are substantially equal.

49. The apparatus of claim 45, wherein the plurality of transducers do not generate acoustic energy while the articles convert previously emitted acoustic energy into electromagnetic energy.

50. The apparatus of claim 45, wherein the processor utilizes the relative conversion efficiencies of the acceptable and unacceptable articles to map their respective locations.

51. A method for differentiating articles, comprising:
generating acoustic energy;
directing the acoustic energy toward a product stream which includes acceptable and unacceptable articles having differing electro-mechanical conversion efficiencies;
converting at least some of the acoustic energy received by the acceptable and unacceptable articles into electromagnetic energy at rates relative to the acceptable and unacceptable articles respective electro-mechanical conversion efficiencies;
detecting at least some of the electromagnetic energy generated by the conversion; and
differentiating the acceptable and unacceptable articles based upon their respective electro-mechanical conversion efficiencies as indicated by the sensed electromagnetic energy.
